(19) United States
(12) Patent Application Publication
(10) Pub. No.: US 2008/0188881 A1
(43) Pub. Date: Aug. 7, 2008

(54) Dual Coil Vitrectomy Probe

(76) Inventor: James Chon, Irvine, CA (US)

Correspondence Address: ALCON, IP LEGAL, TB4-8, 6201 SOUTH FREEWAY, FORT WORTH, TX, 76134, US

(21) Appl. No.: 11/670,766

(22) Filed: Feb. 2, 2007

(51) Int. Cl. A61B 17/32

(52) U.S. Cl. 606/171

(57) ABSTRACT

A vitrectomy probe has a disposable tip portion and a reusable hand piece portion. The disposable tip portion has a shaft terminating in a blade and a sleeve with an opening. The shaft is slideably disposed within the sleeve and capable of reciprocating in the sleeve. The reusable hand piece has a channel for receiving the shaft, first and second coils for driving the shaft in a reciprocating fashion, and a housing enclosing the channel and the first and second coils. The first and second coils are alternately energized to drive the shaft in a reciprocating fashion.

### BACKGROUND OF THE INVENTION

The present invention relates to a vitrectomy probe for use in ophthalmic surgery and more particularly to vitrectomy probe utilizing two coils to produce a fast cut rate.

Anatomically, the eye is divided into two distinct parts—the anterior segment and the posterior segment. The anterior segment includes the lens and extends from the outermost layer of the cornea (the corneal endothelium) to the posterior of the lens capsule. The posterior segment includes the portion of the eye behind the lens capsule. The posterior segment extends from the anterior hyaloid face to the retina, with which the posterior hyaloid face of the vitreous body is in direct contact. The posterior segment is much larger than the anterior segment.

The posterior segment includes the vitreous body—a clear, colorless, gel-like substance. It makes up approximately two-thirds of the eye's volume, giving it form and shape before birth. It is composed of 1% collagen and sodium hyaluronate and 99% water. The anterior boundary of the vitreous body is the anterior hyaloid face, which touches the posterior capsule of the lens, while the posterior hyaloid face forms its posterior boundary, and is in contact with the retina. The vitreous body is not free-flowing like the aqueous humor and has normal anatomic attachment sites. One of these sites is the vitreous base, which is a 3-4 mm wide band that overlies the ora serrata. The optic nerve head, macula lutea, and vascular arcade are also sites of attachment. The vitreous body's major functions are to hold the retina in place, maintain the integrity and shape of the globe, absorb shock due to movement, and to give support for the lens posteriorly. In contrast to aqueous humor, the vitreous body is not continuously replaced. The vitreous body becomes more fluid with age in a process known as syneresis. Syneresis results in shrinkage of the vitreous body, which can exert pressure or traction on its normal attachment sites. If enough traction is applied, the vitreous body may pull itself from its retinal attachment and create a retinal tear or hole.

Various surgical procedures, called vitreo-retinal procedures, are commonly performed in the posterior segment of the eye. Vitreo-retinal procedures are appropriate to treat many serious conditions of the posterior segment. Vitreo-retinal procedures treat conditions such as age-related macular degeneration (AMD), diabetic retinopathy and diabetic vitreous hemorrhage, macular hole, retinal detachment, epiretinal membrane, CMV retinitis, and many other ophthalmic conditions.

A vitrectomy is a common part of a vitreo-retinal procedure. A vitrectomy, or surgical removal of the vitreous body, may be performed to clear blood and debris from the eye, to remove scar tissue, or to alleviate traction on the retina. Blood, inflammatory cells, debris, and scar tissue obscure light as it passes through the eye to the retina, resulting in blurred vision. The vitreous body is also removed if it is pulling or tugging the retina from its normal position. Some of the most common eye conditions that require a vitrectomy include complications from diabetic retinopathy such as retinal detachment or bleeding, macular hole, retinal detachment, pre-retinal membrane fibrosis, bleeding inside the eye (vitreous hemorrhage), injury or infection, and certain problems related to previous eye surgery.

A surgeon performs a vitrectomy with a microscope and special lenses designed to provide a clear image of the posterior segment. Several tiny incisions just a few millimeters in length are made on the sclera at the pars plana. The surgeon inserts microsurgical instruments through the incisions such as a fiber optic light source to illuminate inside the eye, an infusion line to maintain the eye's shape during surgery, and instruments to cut and remove the vitreous body.

The surgical machines used to perform procedures on the posterior segment of the eye are very complex. Typically, such ophthalmic surgical machines include a main console to which numerous different tools are attached. The main console provides power to and controls the operation of the attached tools. The attached tools typically include probes, scissors, forceps, illuminators, vitrectors, and infusion lines. A computer in the main surgical console monitors and controls the operation of these tools.

In a vitrectomy, for example, the vitrector cuts the vitreous body which is then removed through aspiration. Most vitrectors typically use a guillotine action to cut the vitreous body. A cutting blade is disposed within a cannula and is rapidly moved up and down to create a cutting action. A single electric motor is typically used to move the cutting blade. In some vitrectors, the motor propels the cutting blade in one direction and a biasing spring returns the blade to its original position. In other vitrectors, a single electric motor is connected to a rotary mechanism that operates the cutting blade. In both cases, the additional mechanical components—the spring or the rotary mechanism—limit the vitrector's ability to operate at very high cut rates. For example, when a spring is utilized, the electric motor must be operated to overcome the spring force. The spring force governs the maximum cut rate. For a higher cut rate to be achieved, a higher spring force must be used, which in turn requires a higher magnetic flux output by the electric motor. When a rotary mechanism is utilized, vibration may result at higher cut rates. It would be desirable to have a high speed vitrector that utilizes two motor coils to drive the cutting blade without additional mechanical components.

### SUMMARY OF THE INVENTION

In one embodiment consistent with the principles of the present invention, the present invention is a vitrectomy instrument having a disposable tip portion and a reusable hand piece portion. The disposable tip portion includes a shaft terminating in a blade and a sleeve with an opening. The shaft is slideably disposed within the sleeve and capable of reciprocating in the sleeve. The reusable hand piece has a channel for receiving the shaft, first and second coils for driving the shaft in a reciprocating fashion, and a housing enclosing the channel and the first and second coils.

In another embodiment consistent with the principles of the present invention, the present invention is a vitrectomy probe having a disposable tip portion and a reusable hand piece portion. The disposable tip portion has a shaft terminating in a blade and a sleeve with an opening. The shaft is slideably disposed within the sleeve and capable of reciprocating in the sleeve so that the blade reciprocates in the opening. The reusable hand piece has a channel for receiving the shaft, first and second coils for driving the shaft in a reciprocating fashion, and a housing enclosing the channel and the first and second coils. The first and second coils are alternately energized to drive the shaft in a reciprocating fashion.

In another embodiment consistent with the principles of the present invention, the present invention is a vitrector having a disposable tip portion, a reusable hand piece, and a current source. The disposable tip portion has a shaft and a sleeve. The shaft terminates in a blade configured to cut vitreous tissue. The sleeve has an opening through which vitreous tissue may enter the sleeve to be cut. The shaft is slideably disposed within the sleeve and capable of reciprocating in it. The reusable hand piece has a channel for receiving the shaft, first and second coils for driving the shaft in a reciprocating fashion, and a housing enclosing the channel and the first and second coils. The current source provides pulses of current alternately to the first and second coils, thereby energizing the coils to drive the shaft in a reciprocating fashion.

It is to be understood that both the foregoing general description and the following detailed description are exemplary and explanatory only and are intended to provide further explanation of the invention as claimed. The following description, as well as the practice of the invention, set forth and suggest additional advantages and purposes of the invention.

### BRIEF DESCRIPTION OF THE DRAWINGS

The accompanying drawings, which are incorporated in and constitute a part of this specification, illustrate several embodiments of the invention and together with the description, serve to explain the principles of the invention.

FIG. 1 is a perspective view of a vitrector probe including a disposable tip and a re-useable hand piece according to an embodiment of the present invention.

FIG. 2 is an exploded view of a tip portion of a vitrectomy probe according to an embodiment of the present invention.

FIG. 3 is an exploded view of a tip portion of a vitrectomy probe according to an embodiment of the present invention.

FIG. 4 is cross section view of a hand piece utilizing two coils according to an embodiment of the present invention.

FIG. 5 is cross section view of a two coil hand piece and a cutter shaft in an extended position according to an embodiment of the present invention.

FIG. 6 is cross section view of a two coil hand piece and a cutter shaft in a retracted position according to an embodiment of the present invention.

### DETAILED DESCRIPTION OF THE PREFERRED EMBODIMENTS

Reference is now made in detail to the exemplary embodiments of the invention, examples of which are illustrated in the accompanying drawings. Wherever possible, the same reference numbers are used throughout the drawings to refer to the same or like parts.

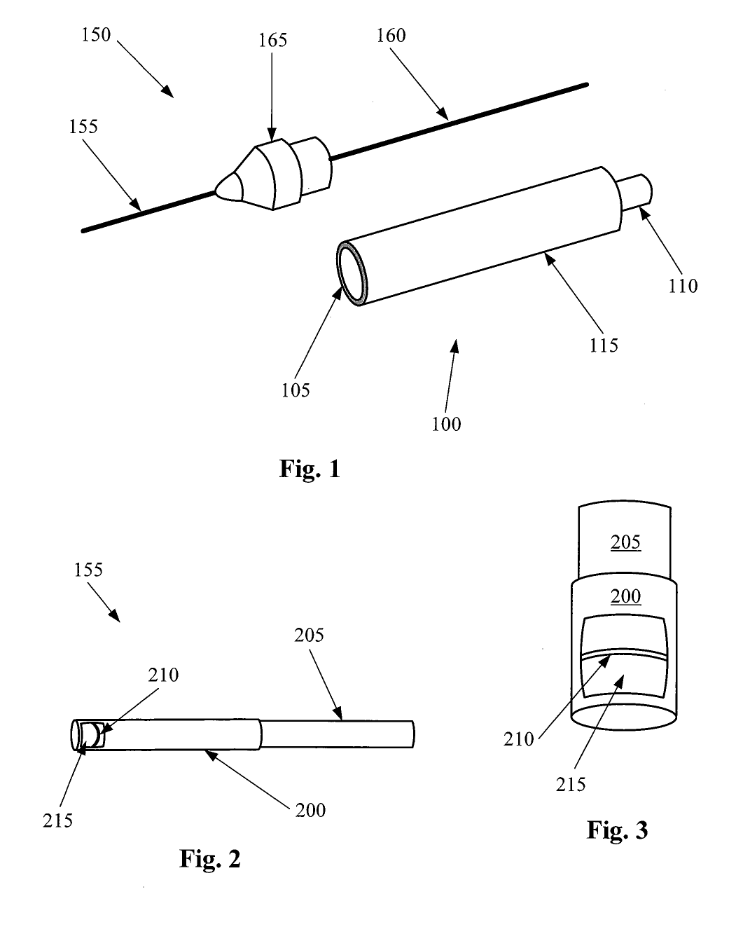

FIG. 1 is a perspective view of a vitrector probe including a disposable tip and a re-useable hand piece according to an embodiment of the present invention. In the embodiment of FIG. 1, hand piece 100 includes housing 115 with proximal and distal ends 105 and 110, respectively. Disposable tip 150 includes probe tip 155, shaft 160, and probe housing 160. Disposable tip 150 is designed to be inserted into proximal end 105 of hand piece 100. Disposable tip 150 is connected to hand piece 100 via a threaded connection or other type of locking connection.

Hand piece 100 is a reusable surgical instrument. Hand piece 100 provides the driving force to operate disposable tip 150. Typically, hand piece 100 is connected by a cable (not shown) to a surgical console that controls its operation. Disposable tip 150 is designed to cut the vitreous during a vitrectomy.

FIG. 2 is an exploded view of a tip portion of a vitrectomy probe according to an embodiment of the present invention. FIG. 2 depicts an embodiment of the probe tip 155. Probe tip 155 includes a sleeve 200 into which a shaft 205 is inserted. Sleeve 200 has an opening 215 near one of its ends. Shaft 205 terminates in a blade 210. Shaft 205 reciprocates within sleeve 200 so as to produce a guillotine cutting action. In operation, vitreous tissue enters opening 215 and is cut by blade 210.

FIG. 3 is an exploded view of a tip portion of a vitrectomy probe according to an embodiment of the present invention. FIG. 3 depicts a cutaway view of sleeve 200 that reveals the placement of shaft 205. As in FIG. 2, shaft 205 terminates in blade 210. Blade 210 moves up and down along with shaft 205 in sleeve 200. In one embodiment, blade 210 travels the entire height of opening 215 to produce a guillotine cutting action. FIG. 3 depicts a location of blade 210 intermediate in opening 215.

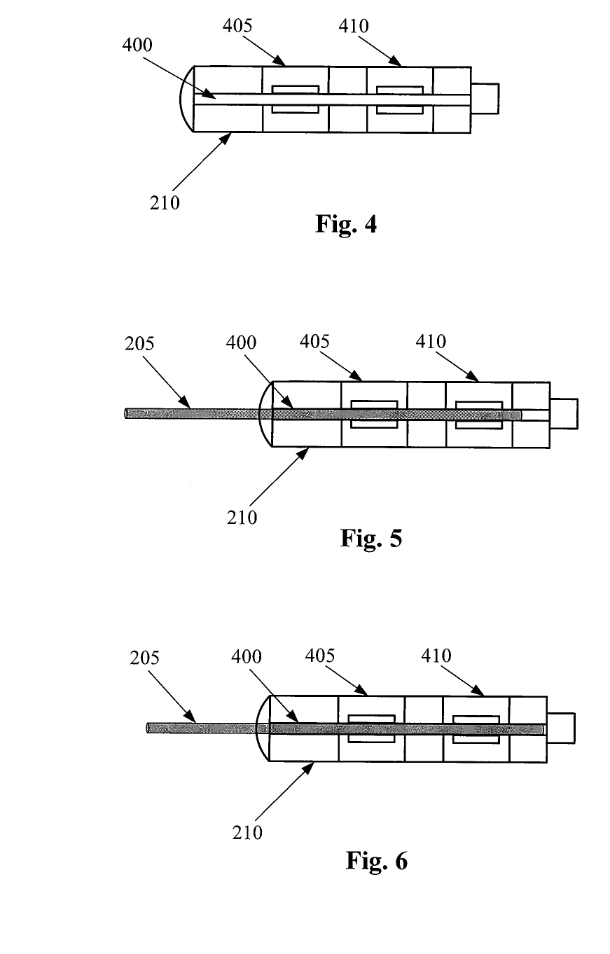

FIG. 4 is cross section view of a hand piece utilizing two coils according to an embodiment of the present invention. In the embodiment of FIG. 4, two coils are utilized to drive the vitrector. Housing 210 encloses a first coil 405, a second coil 410, and a channel 400. First and second coils 405, 410 are disposed around channel 400. Channel 400 is configured to receive vitrector shaft 205 (as shown in FIGS. 5 and 6).

In operation, first coil 405 drives the shaft and attached cutting blade in a first direction, and second coil 410 drives the shaft and attached cutting blade in a second, opposite direction. In this manner, the shaft is reciprocated back and forth to produce a guillotine cutting action. First coil 405 provides a force that moves the shaft in one direction, and second coil 410 provides a force that moves the shaft in the opposite direction. In this manner, only the first and second coils 205, 210 are used to drive the shaft with no additional mechanical components needed.

FIG. 5 is cross section view of a two coil hand piece and a cutter shaft in an extended position according to an embodiment of the present invention. In FIG. 5, shaft 205 is pictured in an extended position. Shaft 205 is disposed in and can slide back and forth in channel 400. In this position, first coil 405 is energized displacing shaft 205 forward. Second coil 410 is not energized.

FIG. 6 is cross section view of a two coil hand piece and a cutter shaft in a retracted position according to an embodiment of the present invention. In FIG. 6, shaft 205 is pictured in retracted position. Shaft 205 is disposed in and can slide back and forth in channel 400. In this position, second coil 410 is energized displacing shaft 205 backward. First coil 405 is not energized.

In operation, current is alternated between the first and second coils 405, 410. When first coil 405 is energized, second coil 410 is not energized. Likewise, when second coil 410 is energized, first coil 405 is not energized. As is commonly known, when current is passed through a coil, a magnetic flux is produced. This magnetic flux exerts a force on shaft 205, at least part of which contains a ferrous material. In one embodiment, shaft 205 is made of stainless steel. The magnetic flux exerted on shaft 205 causes it to move in a direction along channel 400. The two coils 405, 410 are constructed such that, when energized, one coil produces a magnetic flux in one direction and the other coils produces a magnetic flux in the other direction. Alternatively, a positive voltage is applied across one coil, and a negative voltage is applied across the other coil.

The two coil configuration depicted in FIGS. 4-6 allow for high speed operation of the vitrector. Current can be rapidly alternated between the two coils to produce cut rates of 10,000 cuts per minute or more. For example, a current pulse chain in which three millisecond pulses are alternately applied to the two coils can produce a cut rate of about 10,000 cuts per minute. Any number of different current pulse chains can be applied to the coils to provide different operating modes. In addition, the use of two coils without any other mechanical components (like springs or rotary mechanisms) results in smoother operation of the cutter.

From the above, it may be appreciated that the present invention provides an improved system for driving a high speed vitrector. The present invention provides a reusable hand piece with two coils that drive the vitrector. A disposable tip contains the guillotine cutting device. The tip is inserted into the hand piece, and current is alternately applied to the two coils to move the cutter in a reciprocating fashion. The present invention is illustrated herein by example, and various modifications may be made by a person of ordinary skill in the art.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A vitrectomy instrument comprising:
a disposable tip portion including a shaft terminating in a blade and a sleeve with an opening, the shaft slideably disposed within the sleeve and capable of reciprocating in the sleeve; and
a reusable hand piece comprising a channel for receiving the shaft, first and second coils for driving the shaft in a reciprocating fashion, and a housing enclosing the channel and the first and second coils.

2. The instrument of claim 1 in which the first and second coils each surround the channel.

3. The instrument of claim 1 further comprising:
a current source for providing current to the first and second coils.

4. The instrument of claim 3 in which the current source alternately provides current to the first and second coils thereby moving the shaft in a reciprocating fashion.

5. The instrument of claim 4 in which the current source produces a current pulse train.

6. The instrument of claim 4 in which the current source produces a current pulse train of three millisecond pulses to produce a cut rate of about 10,000 cuts per minute.

7. The instrument of claim 1 further comprising:
a voltage source for providing a voltage alternately across the first and second coils.

8. The instrument of claim 1 further comprising a threaded connection between the disposable tip and the hand piece.

9. A vitrectomy probe comprising:
a disposable tip portion comprising a shaft terminating in a blade and a sleeve with an opening, the shaft slideably disposed within the sleeve and capable of reciprocating in the sleeve so that the blade reciprocates in the opening; and
a reusable hand piece comprising a channel for receiving the shaft, first and second coils for driving the shaft in a reciprocating fashion, and a housing enclosing the channel and the first and second coils;
wherein the first and second coils are alternately energized to drive the shaft in a reciprocating fashion.

10. The probe of claim 9 in which the first and second coils each surround the channel.

11. The probe of claim 9 further comprising:
a current source for providing current to the first and second coils.

12. The instrument of claim 11 in which the current source produces a current pulse train.

13. The probe of claim 11 in which the current source produces a current pulse train of three millisecond pulses to produce a cut rate of about 10,000 cuts per minute.

14. The probe of claim 9 further comprising:
a voltage source for providing a voltage alternately across the first and second coils.

15. A vitrector comprising:
a disposable tip portion comprising a shaft and a sleeve, the shaft terminating in a blade configured to cut vitreous tissue, the sleeve having an opening through which vitreous tissue may enter the sleeve to be cut, the shaft slideably disposed within the sleeve and capable of reciprocating in the sleeve;
a reusable hand piece comprising a channel for receiving the shaft, first and second coils for driving the shaft in a reciprocating fashion, and a housing enclosing the channel and the first and second coils; and
a current source for providing pulses of current alternately to the first and second coils, thereby energizing the coils to drive the shaft in a reciprocating fashion.
